(19) United States
(12) Patent Application Publication
(10) Pub. No.: US 2023/0149593 A1
(43) Pub. Date: May 18, 2023

(54) WOUND DRESSING ARTICLES MADE OF ARRAYS OF MICROSCOPIC NEEDLES OBTAINED FROM EXOSKELETONS OF CRUSTACEANS AND METHOD OF MANUFACTURING THE SAME

(71) Applicant: Hiep Thi NGUYEN, Ho Chi Minh (VN)

(72) Inventor: Hiep Thi NGUYEN, Ho Chi Minh (VN)

(73) Assignees: Vietnam National University Ho Chi Minh City, Ho Chi Minh (VN); Ho Chi Minh City International University, Ho Chi Minh (VN)

(21) Appl. No.: 17/814,526

(22) Filed: Jul. 24, 2022

(30) Foreign Application Priority Data
Nov. 17, 2021 (VN) 1-2021-07354

(51) Int. Cl. A61L 15/40; A61M 37/00; A61L 15/44

(52) U.S. Cl. CPC A61L 15/40; A61M 37/0015; A61L 15/44; A61M 2037/0053; A61M 2037/0023

(57) ABSTRACT

The present invention provides a novel and simple method for fabricating a layer (membrane) containing arrays of microscopic needles obtained from crab shells capable of effectively treating wounds and having other medical applications; the method comprises crustacean exoskeletons treated with a base solution, an acid solution, and then medicinally active ingredients such as polysaccharide, proterin, artificial polymer, and inorganic solution.

### CROSS-REFERENCE TO RELATED APPLICATION

This application is based upon and claims priority to a patent application filed in the Republic Socialist of Vietnam. No. 1-2021-07354 entitled “ [symbol] [symbol] [symbol] ” filed in Nov. 17, 2021, the entire content of which is incorporated herein by reference.

### FIELD OF THE INVENTION

The present invention relates generally to the field of medical materials. More specifically, the present invention relates to a medical wound dressing articles made from crab shells.

### BACKGROUND ART

Medical bandages are commonly used for myriad of wound cares. They are used to treat grave wounds such as lymphedema, venous hypertension, leg ulceration; and minor wounds such as cuts and scrapes, etc. In addition, they are designed to inhibit and/or absorb bodily fluids from flowing, such as blood, lymph fluid, or exudate, leading to wound healing.

At the present medical bandages are available in a variety of types, however, they all have drawbacks in usage and in manufacturing processes. They cannot accelerate the regrowth of new epidermis skins and promote the healing process to wounds. Antibotic gauzes are medical bandages treated with antibiotic materials such as silver (Ag), iodine (I), chlorhexidine gluconate (CHG), etc. This type of gauze has the capability of antiviral, decreasing or preventing infection. Antibiotic gauzes are usually used for grave, infected, and seromatic wounds. However, they cannot be used for patients who are allergic to these materials.

Collagenic gauzes are made in different forms such as bandages, powder, gel which are extracted from skins and tissues of bovine buffaloes, horses, fowls, and porcupines. These gauzes speed the condensation and formation of collagen fibers, new tissues in the wounds, and accelerating the wound healing process. However, collagen gauzes are limited in use only with surgery wounds which are built up with serum, infected, chronic, and vascular.

Hydrogel gauzes help easing the pains, maintaining moisture to the wounds, accelarting the process of forming new tissues, new granules (stratum granulosum), and epidermis layer. These types of gauzes are often used in wounds with less serum, infected, light burn, and scraped wounds. They are not used in wounds with serum build-up because these gauzes contain a definite amount of fluid and therefore, they cannot absorb more fluids.

The fibrous gel gauzes are made of sodium carboxymethyl cellulose (CMC). These types of gauzes have the capability of maintaining, controlling the seroma in the wounds, and eliminating infected or dead tissues. The seroma—when absorbed into the gauzes—will form a layer of gel that helps maintaining a moist surrounding for the wounds, thus facilitating the growth of granular layers, and thus promoting the healing of the wounds. These types of gauzes are used in serious and chronic wounds, burnt, and ulcers. Again, these types of fibrous gel gauzes are not used with people who are allergic to them.

Nonwoven cloth gauzes are made of synthetic fabrics such as cotton, polyesters, or artificial fibers that are pressed together. As such they have better absorbing capability than woven fabrics. These types of gauzes are used in shallow, dry with little seroma wounds. When gauzes are removed, it is recommended to use biological salts to wet the gauzes before taking it off to avoid damaging the wounds because the tissues of the gauzes have been stuck with those of the wounds.

Porous gauzes are mostly made from polyurethane, a hydrophilic material cable of strong absorbing and retaining fluids. They can be absorbed or coated with other materials depending on functional uses. This type of gauze does not stick to the wound. They are easily removed without causing any pains. Porous gauzes are used in wounds replete with seroma, chronic, relatively shallow. They are not used with dry wounds with no seroma and third-degree burning wounds.

Alginate gauzes are made from nonwoven calcium algenate fibers extracted from brown sea algae. They have different forms such as patches or fibers. This type of gauzes has the absorbing capability of 15-20 times more than the weight of the gauze itself. When contact with the seroma of the wounds, these gauzes form a biological gel layer that maintains the moist surrounding and enkindle the granular tissues to grow, helping the wound heal faster. Alginate gauzes are used in chronic wounds that excrete a lot of serum, liquefied fat, and lymphatic fluid. Therefore, these types of gauzes are not used in dry wounds, surgery wounds, and third degree burn wounds.

The hydrocolloid gauzes are made from gelatin, pectin, polysaccharide, or sodium carboxymethyl cellulose (CMC) with different forms such as powder, gel, or patches (band-aid). The special characteristics of this type of gauze is that when contacting with the wounds, the constituents will form a gel layer with the serenum of the wound that help maintaining the moisture ideal for the healing process and preventing the multification of the micro-organisms. These gauzes are used in clean, dry, less seronum, average depth such as abrasions or chronic wounds in the process of growing granular tissues and forming new epidermis skin layer. These gauzes cannot be used in wounds with seronum, burn wounds, infected wounds, thick, deep, with many grooves and subgrooves.[2]

As described above, the existing medical bandages have limited uses and they are slow or incapable of healing wounds. Recently, there existed medical bandages made from natural resources such as chitin/chitosan from the carapaces of crustaceans such as crabs.

Chitosan is more commonly used than chitin in making medical bandages, tapes, and dressing that heal external wounds and enkindle the growth of the new skins that accelerate wound healing. These medical bandages, tapes, and dressing are synthesized from the hard shells of crustacean such as crabs, shrimps, lobsters, and mollusks such as snell, clamps, etc. which are soaked in special solvents in order to obtain an extract rich in chitin materials. In the conventional process, the chitin and chitosan materials are obtained using a complex extraction process. The extraction is either mixed with the foundational material such as single piece nonwoven cloths to make bandages or weaved into fibers that are shaped into various dressing and bandages depending on the needs or added with materials to make wound dressings in form of gel, hydrogel. To obtain chitosan, chitin extracted from crab shells had to be deacetylized first. Deacetylizing means removing acetyl-group (CH₃CO) from chitin molecules. However, deacetylating chitin to chitosan requires intensive energy, generates concentrated alkaline waste, and produces a broad range of soluble and insoluble products that are challenging to process before being released into the environment [4]. In one prior-art approach, Jingping Zhou and his team have created medical gauze made from chitin fibers. His group found that dressings made from chitin with 71 percent of acetylated glucose worked best of all and would speed the healing process. In the labs, Jingping Zhou used chitin from crab shells to test out new medical bandages. The crab shells were first ground into bits and then went under a process of soaking the gritty bits in special solvent for 12 hours followed by heating, bleaching, and a complex process to turn the chitin rich solution into moist fibers [1]. As described above, the Jingping Zhou's approach demanded a complex process to obtain the moist fiber. However, the moist fiber obtained from the Jingpin zhou approach was the chitin chemical material itself which had been used as medical materials since 1970. The Jingping Zhou's approach and the conventional approach are focusing on the chitin chemical material and not the membrane from the crab shells.

Therefore, what is needed is a novel medical materials and medical gauze and that are simple and inexpensive to manufacture and effective in healing a broad range of wounds ranging from simple scratches or cuts to serious wounds for the gravely ill people such as diabetic patients.

What is needed is a method of manufacturing medical materials that are biocompatible and can bind to the wound tissues and deliver antibiotic materials to the wound.

What is needed is a medical gauzes and method of manufacturing the same that focus on the wholesome membrane elicited from the crab shells instead of extracting chitin and chitosan materials therefrom through complex process.

What is needed is a medical gauzes and method of manufacturing the same that combine natural resources such as raw chitin membrane from crab shells and polymeric substrate that produce medical gauzes effective in healing wounds.

The present invention provides solutions to the above problems and meets the long-felt needs in new medical materials and the medical wound dressings.

### SUMMARY OF THE INVENTION

Accordingly, an object of the present invention is to provide novel medical materials such as a layer of arrays of microscopic needles extracted from the interior side of crab shells obtained from the process of deproteinization and demineralization at precise temperature, concentration, and duration.

An object of the present invention is to provide wound dressing articles comprised of crab shells treated with a base solution and an acid solution and of a medicinally active ingredients including polysaccharides, artificial polymer, and protein mixture.

Another object of the present invention is to provide a method of manufacturing medical dressing articles including the steps of (a) preparing soft crustacean shells (exoskeletons) having a first predetermined percentage weight (% w/w); treating the soft crustacean shells with a base solution at a temperature of 25° C. to 50° C. for 6 hours to 12 hours; (c) treating the soft crustacean shells with an acid solution at a temperature of 25° C. to 50° C. for 6 hours to 12 hours; and (d) adding medically active ingredients—having a second predetermined percentage weight (% w/w)—comprised of polysaccharide, artificial polymer, and protein.

Yet another object of the present invention is to provide medical dressing articles made from natural products that are biologically comparable to wounds to accelerate the regrowth of new epidermis skins for various types of wounds, promote the healing process to open wounds, and at the same time have the capability to absorb seroma fluids from other wounds, and prevent infections.

These and other advantages of the present invention will no doubt become obvious to those of ordinary skill in the art after having read the following detailed description of the preferred embodiments, which are illustrated in the various drawing Figures.

### BRIEF DESCRIPTION OF THE DRAWINGS

The accompanying drawings, which are incorporated in and form a part of this specification, illustrate embodiments of the invention and, together with the description, explain the principles of the invention.

FIG. 1 is a method of preparing a layer (membrane) containing arrays of microscopic needles obtained from exoskeletons of crustaceans in accordance with an exemplary embodiment of the present invention.

FIG. 2A-2B are images taken by a scanning electron microscope (SEM) magnified at 100 times and 500 times comparing the internal and external sides of crab shells before and after being treated with base solution and then acid solution with an exemplary embodiment of the present invention.

FIG. 3 is a method of fabricating a medical dressing using a polymeric substrate of polycaprolactone and/or poloxamer and the layer (membrane) containing arrays of microscopic needles obtained from exoskeletons of crustaceans in accordance with an exemplary embodiment of the present invention.

FIG. 4A-FIG. 4C show different structures of medical dressing articles made of the polymeric substrate of polycaprolactone and/or poloxamer and the layer (membrane) containing arrays of microscopic needles obtained from exoskeletons of crustaceans in accordance with various embodiments of the present invention.

FIG. 5 is a diagram showing the medical dressing article with the layer (membrane) containing arrays of microscopic needles making directly contact with a wound in accordance with various embodiments of the present invention.

FIG. 6 is a graph of the elasticity and the strength of medical dressing articles using the layer (membrane) containing arrays of microscopic needles obtained from exoskeletons of crustaceans in accordance with an exemplary embodiment of the present invention.

FIG. 7 is graphs comparing the spectrum of a FT-IR Fourier Transform Infrared Spectroscopy of (a) raw untreated crab shells, (b) crab shells treated with a base solution and (c) crab shells treated with acid solution.

FIG. 8 is a bar graph showing the survivability of cells in the medical dressing articles made of crab shells treated with a base solution and an acid solution at different concentration of 0%, 12.5%, 25%, 50%, and 100%.

FIG. 9 are SEM images showing the survivability of cells found in a wound with and without the treatment of the medical dressing articles of the present invention in different time periods of one hour, one day, and seven days.

FIG. 10A is a graph showing the healing rates of a wound treated with the medical dressing article of the present invention as compared to an untreated wound during a time duration of 14 days.

FIG. 10B is an image showing different layers of normal and healthy skin.

FIG. 11 shows images of hematoxylin & Eosin (H&E) of wounds treated with the medical dressing article of the present invention as compared to untreated wounds in the duration of one week and two weeks.

### DETAILED DESCRIPTION OF THE INVENTION

Reference will now be made in detail to the preferred embodiments of the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications, and equivalents, which may be included within the spirit and scope of the invention as defined by the appended claims. Furthermore, in the following detailed description of the present invention, numerous specific details are set forth in order to provide a thorough understanding of the present invention. However, it will be obvious to one of ordinary skill in the art that the present invention may be practiced without these specific details. In other instances, well-known methods, procedures, components, and circuits have not been described in detail so as not to unnecessarily obscure aspects of the present invention.

As used herein, the term “medical dressing articles” refers to bandages, gauzes, band-aids, cloths, pads, plasters, tapes, or the likes designed to cover external wounds regardless of the forms such as gels, gelatins, unwoven cloths, etc.

As used herein, the term “shells” refers to exoskeletons, carapaces which are bony or chitinous shield, test, or shell covering some or all of animal as of crustaceans such as crabs, lobsters, shrimps; porcupines; turtles, fowls such as roosters, buffaloes, and cows.

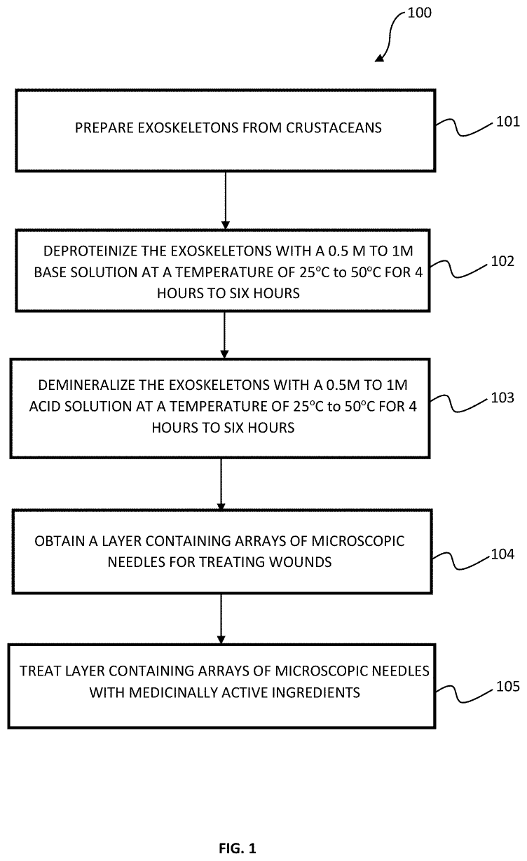

Many aspects of the present invention are now described with reference to FIG. 1-FIG. 11. A medical dressing article of the present invention includes a layer (membrane) containing arrays of microscopic needles elicited from the interior side of crab shells after being treated with a base solution and then an acid solution at precise predetermined condition. More particularly, in order to obtain such layer containing arrays of microscopic needles, the crab shells must be carefully treated with the base solution includes sodium hydroxide (NaOH), potassium hydroxide (KOH), or other bases having a base molarity concentration between 0.5M to 2M for 4 to 6 hours at a temperature between 25° C. to 50° C.; then with the acid solution includes hydrochloric acid (HCl) or other acid solutions with an acid molarity concentration between 0.5M to 2M for 4 to 6 hours at a temperature between 25° C. to 50° C. When soft crab shells from molting crabs are used, the molarity concentration of base solution and acid solution is from 0.5M to 1 M and 0.5M to 1 M respectively. The layer containing arrays of microscopic needles was proven to bind with cells and tissues in wounds, epithelizing wounds tissues and delivering medicinal ingredients for faster healing process.

Now referring first to FIG. 1, a method 100 for preparing a layer of crustacean exoskeleton that contains arrays of microscopic needled that are used to make medical dressing articles in accordance with an exemplary embodiment of the present invention is illustrated. In order to achieve the layer (membrane) containing arrays of microscopic needles layer of the present invention, exoskeletons of crustaceans are first deproteinized by treating with a base solution having specific concentration for a specific time duration and at a specific temperature; then the deproteinized exoskeletons of crustaceans are demineralized by treating with a base solution having specific concentration for a specific time duration and a specific temperature. Afterwards, the layer having arrays of microscopic needles is obtained and used in making medical dressing articles and other applications. Method 100 for preparing the layer having arrays of microscopic needles briefly described above includes the following steps:

At step 101, crustacean exoskeletons such as crab shells are prepared. In preferable embodiments of the present invention, crustacean exoskeletons of step 101 are crab shells. These crab shells may be from any crabs purchased in the market. In some embodiments of the present invention, crab shells may be selected from soft shells on molting crabs or freshly discarded shells from molting crabs. Molting is a process where crabs outgrow their old shells, they shred the old shells, and grow new soft shells. If brand new soft shells grown on molting crabs are used, a pair of scissors are used to separate the new soft shells from the crabs. The new soft shells are then washed thoroughly with distilled water to get rid of dirts, particulates, and impurities. The cleaned new soft crab shells are dried in the sun. In case when the discarded shells are used, after being collected from their farming ponds, muds and dirts are first washed off. Next, the discarded shells are washed with distilled water and dried in the sun.

At step 102, the cleaned crab shells are deproteinized by treating them with a 0.1M to 2M base solution at a temperature of 25° C. to 50° C. for 4 hours to 6 hours. Inside the base solution, the cleaned crab shells are stirred thoroughly to remove protein therefrom. This is because proteins in the crab shells tend to react with wounds, providing foods for viruses or bacteria. Step 102 is known as deproteinization. Deproteinization deprives pathogenic microorganism in wounds of foods such as proteins. Afterwards, the base treated crab shells, also known as deproteinized crab shells, are washed with distilled water three times, and let dry in room temperature. In many aspects of the present invention, strong base such as sodium hydroxide (NaOH), potassium hydroxide (KOH), or other alkaline base solution with similar chemical characteristics thereof with molarity concentration between 0.5M to 2M are used.

Next, at step 103, the deproteinized crab shells in the previous step 102 are next demineralized by treated them with an acid solution at a temperature of 25° C. to 50° C. for 4 hours to 6 hours. Step 103 is also known as demineralization process designed to remove the deproteinized crab shells from minerals such as calcium carbonate (CaCO₃). Minerals in the crab shells may adversely react with other elements within the wounds. In various aspects of the present invention, acid solution used is hydrochloric acid (HCl) or similar acid with the same chemical characteristics having a molarity concentration between 0.5M to 2M.

Afterwards, at step 104, the deproteinized and demineralized crab shells are washed three times with distilled water and let dry out at room temperature to obtain a layer having arrays of microscopic needles (hereinafter referred to as “microscopic needles layer”). The properties of the microscopic needles layer of the present invention lie in many fundamental physiological processes: in fact, the microscopic needles layer separates tissues and organs, and is responsible for their mechanical properties; it maintains tissue homeostasis because of its buffering action and water retention ability. Furthermore, the microscopic needles layer provides substrate for cell anchorage and motility, and it works as a reservoir of biochemical factors important for cellular functions. Finally, the microscopic needles layer is proven to regulate cell communications and could influence cell behavior in several ways, both during tissue morphogenesis, homeostasis and wound healing. Finally, the microscopic needles layer functions are usually mediated by cell-ECM adhesions.

Next, at step 105, the microscopic needles layer elicited from the crab shells are mixed with medicinally active ingredients comprised of polysaccharide, protein, artificial polymer, and inorganic elements in order to create the medical dressing article of the present invention. Step 104 is realized by dipping, soaking, or spraying the arrays of microscopic needles with the described medicinally active ingredients. The thickness of the microscopic needles layer is between 0.5 mm to 1 mm. The medicinally active ingredients have a thickness between 10 μm to 100 μm. The relative weight of the medicinally active ingredients is from 0.5% to 5 percent of the weight of the microscopic needles layer. In many aspects of the present invention, the polysaccharide includes hyaluronic acid (C₁₄H₂₁NO₁₁)n, chondroitin sulfate (C₁₃H₂₁NO₁₅S), dextran H(C₆H₁₀O₅)xOH, alginate (C₆HO₆)n, and heparin (C₁₂H₁₉NO₂₀S₃), which are components of skins and therefore they will not react with other elements of the skins, and help antibiotic, antifungal, and antibacterial. The protein group includes sericin (silkworm), fibrin (factor Ia), fibrinogen (protein synthesized in the liver), collagen (structural protein), gelatin (derived from collagen), and gelatin methcryloyl (GelMA, hydrogels), which are all found in human skins, therefore biocompatible to skins and help skin to heal faster. The medicinally active inorganic ingredients include zinc oxide (ZnO), copper oxide (CuO), copper alloy, nano zinc, nano gold, nano copper, and nano aluminum providing antibacterial nano elements to the wounds.

Continuing with step 105, the medicinally active artificial polymer ingredients play a role in the bio compatible that transform the physical characteristics of the membrane from soft to hard include PCL-polycaprolactone, poly(L-lactic acid) (PLLA-poly(L-lactic acid)), poly (L-lactic acid-coglycolic acid) (PLGA-poly(L-lactic acid-co-glycolic acid)), poyetyle glycol (PEG-polyethyl glycol), polyvinyl alcohol (PVA-polyvinyl alcohol), polyaniline (PANI-polyaniline), polyethylenimine (PEI-polyethylenimine).

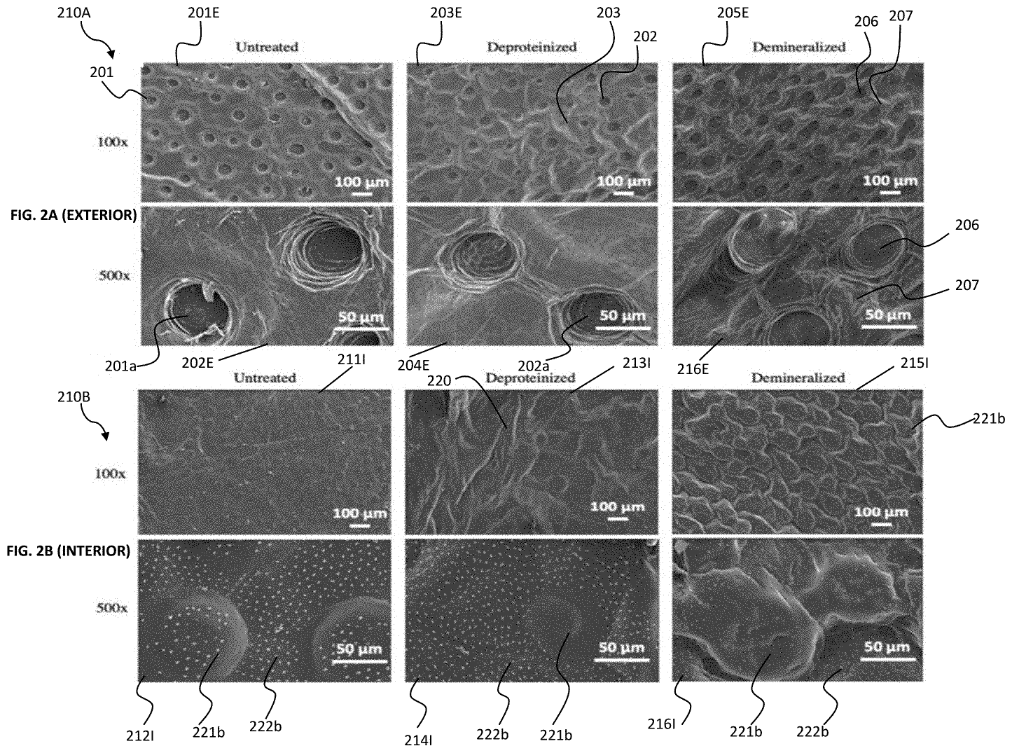

Method 100 of the present invention described above achieves the following objectives: the microscopic needles layer (membrane) obtained from crab shells that can be used to treat wounds and other medical applications such as wound dressing articles. Next, FIG. 2A to FIG. 2B describe the properties of the microscopic needles layer. Method 100 of the present invention is not focused on extracting chitin/chitosan fibers (a bio-chemical compound) to make medical dressing articles. Instead, method 100 includes simple and precise steps of obtain a wholesome membrane containing arrays of microscopic needles from the inner side of crab shells after being washed, deproteinized, and dematerialized. The objective of method 100 is to produce a wholesome membrane that has medical properties suitable to cure different types of wounds and to make medical dressing articles.

Next referring to FIG. 2A, scanning electron microscope (SEM) images 200A of the exterior surface of a crab shell in three different conditions (untreated, deproteinized, and demineralized) and in two different magnifications 100 and 500 are presented. A micrograph 211E illustrates the structure of cleaned and untreated crab shells in step 101 magnified at 100 times. A micrograph 212E illustrates the structure of cleaned and untreated crab shells in step 101 magnified at 500 times.

Micrograph 211E shows the exterior surface of an untreated crab shell magnified by a SEM at 100 times. As shown, a highly hierarchically ordered arrays of mesopores 201a are revealed. Mesorpores 201a are interspacing voids of about 70 nm. A micrograph 212E shows the same mesopores 201a now seen as deep pores or holes at 500 times magnification.

Next, a micrograph 213E illustrates the structure of deproteinized crab shells in step 102 magnified at 100 times. A micrograph 214E illustrates the structure of deproteinized crab shells in step 102 magnified at 500 times.

Micrograph 213E shows that mesopores 201a start to reduce in depth and size and an array of nanofibers 202a start to appear. Micrograph 214E shows mesopores 201a and array of nanofibers 202a on the exterior surface of deproteinized crab shell magnified at 500 times. As shown, mesopores 201a are lighter signifying that their depth was reduced. The sizes of mesopores 201a were reduced and replaced by curvy array of nanofibers 202a.

Followings are a micrograph 215E illustrates the structure of dematerialized crab shells in step 103 magnified at 100 times. A micrograph 216E illustrates the structure of dematerialized crab shells in step 103 magnified at 500 times.

Micrograph 215E shows that after being demineralized with strong acid solution, curvy array of nanofibers 202a appear even clearer in SEM image magnified by 100 times. In micrograph 216E, SEM image of interior surface of demineralized crab shells magnified 500 times shows arrays of nanofibers 202a were proven to provide durability to the needles layer obtained by method 100 described above.

Now referring to FIG. 2B, scanning electron microscope (SEM) images 200B of the interior surface of a crab shell in three different conditions such as untreated, deproteinized, and demineralized and in two different magnifications are presented. A micrograph 2111 illustrates the structure of cleaned and untreated crab shells in step 101 magnified at 100 times. A micrograph 2121 illustrates the structure of cleaned and untreated crab shells in step 101 magnified at 500 times.

Micrograph 2111 shows the interior surface of untreated crab shells which are rather smooth. Micrograph 2121 shows lumps 221b of the interior surface of the untreated crab shells magnified at 500 times. At this magnification scale, lumps 221b are revealed.

Next, a micrograph 2131 illustrates the structure of deproteinized crab shells in step 102 magnified at 100 times. A micrograph 2141 illustrates the structure of cleaned and untreated crab shells in step 102 magnified at 500 times.

More particularly, micrograph 2131 shows fibrorous patterns 220b while micrograph 2141 presenting SEM that shows that a microscopic needle 221b starts to appear as a substrate 222b starts to recede in the interior surface of the deproteinized crab shells magnified at 500 times.

Finally, a micrograph 2151 illustrates the structure of demineralized crab shells in step 103 magnified at 100 times. A micrograph 2161 illustrates the structure of demineralized crab shells in step 102 magnified at 500 times.

Micrograph 2151 presenting SEM image that clearly shows arrays of microscopic needles 221b as alluded above. At the magnification scale of 500 times, an SEM micro image 2161 shows a close-up view of microscopic needles 221b and substrate 222b. These microscopic needles 221b act as prickles that grasp to the wounds and provides scaffolds for cells and tissues to regrow. These microscopic needles 221b has a diameter of 70 μm.

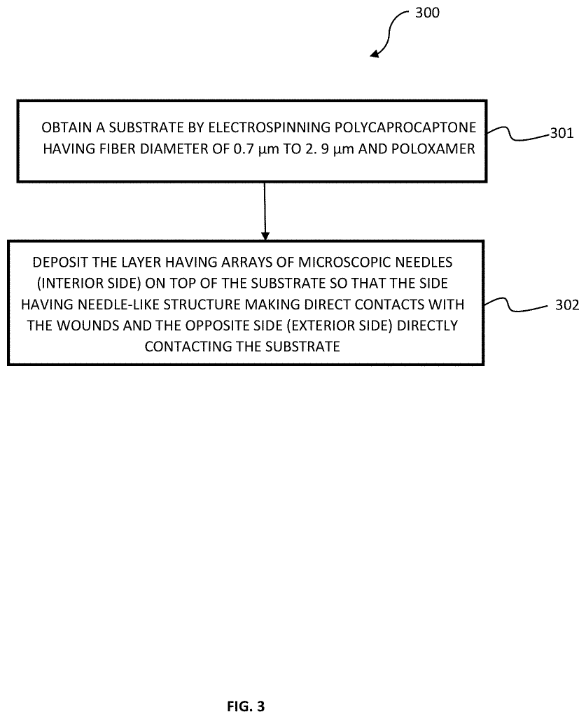

As shown in FIG. 2A-FIG. 2B, morphological changes in the crab shells in both exterior side and exterior side in three different conditions of untreatment, deproteinized, and demineralized are shown. The present invention uses the medical advantages of microscopic needles layer 221b elicited in the interior side of crab shells after being treated with the base solution and the acid solution as described in method 100 above. Next, the needles layer is deposited on a polymeric substrate to obtain a medical dressing article in accordance with an exemplary embodiment of the present invention as described in FIG. 3 below.

Now referring to FIG. 3, a method 300 of making a medical dressing article using the needles layer in accordance with an exemplary embodiment of the present invention is illustrated. It is noted that method 300 is a non-limiting application of layer of microscopic needles layer 221b in fabricating medical wound dressings.

At step 301, a polymeric substrate is selected, and the electrospinning is set up. In many preferred embodiments of the present invention, step 301 is achieved by mixing polycaprolactone (PCL) and poloxamer with a concentration between 10% to 15% by an electrospinning (ES) apparatus operating at a voltage potential between 8 kV to 20 kV. The Taylor cone of the ES is chosen to generate a PCL fiber diameter between 0.7 μm to 2.9 μm. The ration between poloxamer and PCL is respectively between 1:1 to 1:50 and the rate of spinning is 1 mL/hour.

Next, at step 302, the wholesome microscopic needles layer obtained from method 100 of the present invention is deposited on the polymeric substrate of step 301. In many aspects of the present invention, the electrospinning set up as described in step 301 is used with the microscopic needles layer as the collector. The polymeric substrate is spun on the exterior side (see 215E and 216E in FIG. 2A) of crab shell after step 203 is completed. The microscopic needles layer (see 2151 and 2161 in FIG. 2B) where the microscopic needles are configured to face outward to make direct contact with the wounds. As alluded above, step 302 involves a wholesome layer of arrays of microscopic needles from the interior side of crab shells. This is novelly different from the prior-art medical dressing articles which focus mainly on the chitin/chitosan biomaterials extracted from complex processes.

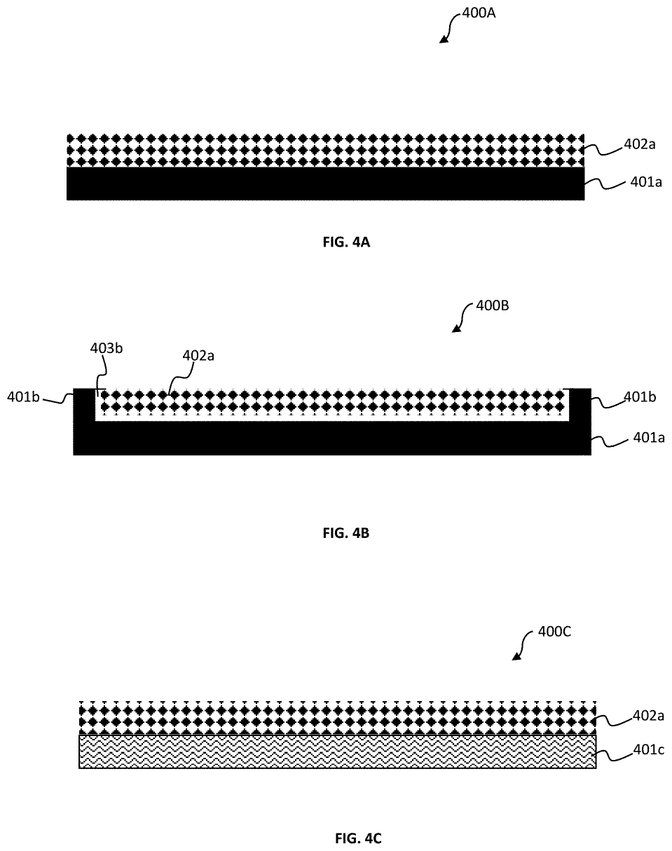

Next referring to FIG. 4A-4C, different structures of medical dressing articles 400A-400C made of the polymeric substrate in combination with a layer containing arrays of microscopic needles layer obtained from exoskeletons of crustaceans in accordance with various embodiments of the present invention are shown. In FIG. 4A, a medical dressing article 400A is comprised of a layer of microscopic needles 402a (layer or membrane 402a) obtained by method 100 and as shown in FIG. 2A-FIG. 2B. As disclosed in FIG. 2, layer 402a includes exterior surface 210A (see FIG. 2A) and interior surface 210B (see FIG. 2B). Exterior surface 210A is deposited directly on an impermeable polymeric substrate such as polycaprolactone (PCL) 401a. In this embodiment, substrate 401a is wound by electrospinning apparatus (ES) on the exterior surface 210A. The Taylor cone of the ES is chosen to generate a PCL fiber diameter between 0.7 μm to 2.9 μm. The rate of spinning is 1 mL/hour. The interior surface 210B as shown in FIG. 2B of layer 402a containing arrays of microscopic needles 221b is of medical importance. Interior surface 210B is arranged to make direct contacts with the wound.

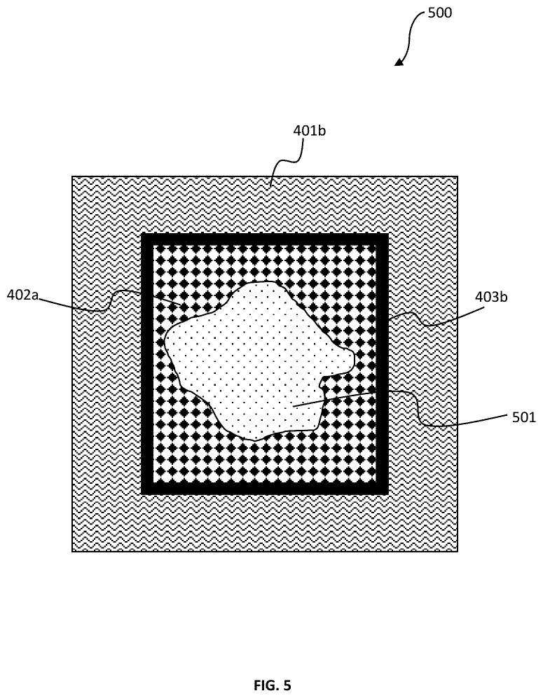

Continuing with FIG. 4B, a medical dressing article 400B with edge protectors 401b is comprised of layer 402a containing arrays microscopic needles 404B as described in FIG. 2A-FIG. 2B. In this embodiment, layer 402a is deposited directly on impermeable polymeric substrate such as polycaprolactone (PCL) 401a by electrospinning apparatus (ES) on the exterior surface 210A. Edge protectors 401b are deposited around the perimeter of layer 402a to protect layer 402a. Please see also FIG. 5. The Taylor cone of the ES is chosen to generate a PCL fiber diameter between 0.7 μm to 2.9 μm. The rate of spinning is 1 mL/hour. Interior surface 210B is allowed to make direct contacts with the wound.

Continuing with FIG. 4C, a medical dressing article 400C is comprised of layer 402a deposited directly on an impermeable polymeric substrate 401c such as polycaprolactone (PCL) mixed with and poloxamer. In this embodiment, substrate 401c is wound by electrospinning apparatus (ES) on exterior surface 210A of the crab shells as shown in FIG. 2A layer 402a. In this embodiment of the present invention, polycaprolactone (PCL) is mixed with poloxamer at a concentration between 10% to 15% by an electrospinning (ES) apparatus operating at a voltage potential between 8 kV to 20 kV. The Taylor cone of the ES is chosen to generate a PCL fiber diameter between 0.7 μm to 2.9 μm. The ratio between poloxamer and PCL is respectively between 1:1 to 1:50. The rate of spinning is 1 mL/hour. The interior surface 210B as shown in FIG. 2B of layer 402a containing arrays of microscopic needles 221b is arranged to make direct contacts with the wound. Again, layer 402a containing arrays of microscopic needles 221b is not chitin/chitosan biomaterials extracted by complex methods. Chitin/chitosan biomaterials used in prior-art medical dressing articles are in form of fiber or powder. They did not have arrays of microscopic needles 221b of the present invention

Referring now to FIG. 5, a diagram 500 illustrating medical dressing article 400B with the layer containing arrays of microscopic needles making directly contact with a wound in accordance with various embodiments of the present invention is shown. Diagram 500 shows a wound 501 treated with medical dressing article 400B with layer 402a deposited directly on polymeric PLC substrate 401a with edge protector 401b.

FIG. 1 to FIG. 5 of the present invention achieves the following objectives:

(1) wound dressing articles comprised of crab shells treated with a base solution and an acid solution and of a medicinally active ingredients including polysaccharides, artificial polymer, and protein mixture.

(2) a method of manufacturing a wholesome membrane containing arrays of microscopic needles with many medical applications, the method including the steps of (a) preparing soft crustacean shells (exoskeletons) having a first predetermined percentage weight (% w/w); treating the soft crustacean shells with a base solution at a temperature of 25° C. to 50° C. for 6 hours to 12 hours; (c) treating the soft crustacean shells with an acid solution at a temperature of 25° C. to 50° C. for 6 hours to 12 hours; and (d) adding medically active ingredients—having a second predetermined percentage weight (% w/w)—comprised of polysaccharide, artificial polymer, and protein.

(3) medical dressing articles made from natural products that are biologically comparable to wounds to accelerate the regrowth of new epidermis skins for various types of wounds, promote the healing process to open wounds, and at the same time have the capability to absorb seroma fluids from other wounds, and prevent infections.

(4) medical dressing articles comprised of a layer (membrane) containing arrays of microscopic needles and polymeric compound such as polycaprolactone (PCL) mixed with and poloxamer.

Experiments to Obtain the Microscopic Needles Layer of the Present Invention

First Experiment: Preparing the Soft Shell Crabs

In this experiment, soft-shell or molting crabs were obtained from a local market, and their soft shells were extracted with scissors. This is an implementation of step 101 described above.

The shells were deproteinized in 1 mole (M) of NaOH at 50° C. for 5 hours, rinsed three times with distilled water, and dried overnight at room temperature to obtain deproteinized soft shells. This is an implementation of step 102 of method 100.

Next, the deproteinized soft shells were then demineralized in 0.5% w/w hydrochloric acid (HCl) for 4 hours, rinsed three times, and dried overnight at room temperature. This is an implementation of step 103. Then, needles layer is obtained by washing the deproteinized and dematerialized crab shells. This is an implementation of step 104.

Second Experiment to Fourth Experiment: Medical dressing articles by adding medicinally active ingredients containing a polysaccharide group comprised of hyaluronic acid (C₁₄H₂₁NO₁₁)ₙ, chondroitin sulfate, and heparin (C₁₂H₁₉NO₂₀S₃) to the Soft Shells. This is an implementation of step 105 of method 100.

Second Experiment: Hyaluronic acid with a concentration of 0.1% to 1% was prepared. The treated shells described above was submerged in the hyaluronic acid for 5 to 10 minutes at a temperature from 4° C. to 20° C. The shells soaked with the hyaluronic acid was obtained. The medical dressing article was obtained having a thickness of 0.5 mm to 1 mm in which the thickness of the hyaluronic acid was 5 μm to 15 μm. The weight of the hyaluronic acid is 0.5% to 2% w/w relative to that of the shells.

Third Experiment: Chondroitin sulfate solution with a concentration of 0.1% to 1% was prepared. Then, the chondroitin sulfate was sprayed onto the shells treated with they hyaluronic acid solution above at a speed of 50 ml/minute in one hour at the temperature from 25° C. to 100° C. and obtained the shells containing chondroitin sulfate. After this step, the medical dressing article (“the article”) has a thickness from 0.5 mm to 1 mm; the chondroitin sulfate has a thickness of 5 μm to 15 μm. The amount of chondroitin sulfate contained in the article at this stage is 0.5% to 2% (% w/w) relative to that of the shells.

Fourth Experiment: Next the heparin solution with a concentration of 2.75% was prepared. Then the article obtained from the steps above was submerged into this heparin solution at a rate of 5 mm/minute in one hour at the temperature of 25° C. to 100° C. The result is the article covered with heparin solution. At this stage, the article has a thickness of 0.5 mm to 1 mm. The thickness of the heparin solution is 5 μm to 15 μm. The relative weight of the heparin solution is 0.5% to 2% (% w/w) relative to that of the shells.

Fifth to Seven Experiments: Adding protein containing sericin solution, collagen, and fibrinogen to the article with heparin solution.

Fifth Experiment: The sericin solution with a concentration of 2% was prepared. The article covered with heparin was submerged in the sericin solution in 10-60 minutes at the room temperature. The article containing heparin was obtained because of this step. The article has a thickness of 0.5 mm to 1 mm. The thickness of the sericin is from 5 μm to 10 μm. The relative weight of the sericin to that of the shells was 0.1% to 0.5% (% w/w).

Sixth Experiment: The collagen solution with a concentration of 0.8% was prepared. Then, the collagen solution was sprayed onto the article with sericin at a speed of 0.5 ml/minute in one hour at the room temperature. After that, the article with collagen was obtained. The thickness of the article after this step is 0.5 mm to 1 mm. The thickness of the collagen solution is 5 μm to 10 μm. The relative weight of the collagen to that of the shells was 0.1% to 0.5% w/w.

Seventh Experiment: The fibrinogen solution with a concentration of 10,000 ppm to 20,000 ppm was prepared. The article with collagen above was covered with fibrinogen solution with a dipping rate at 1 mm/minute in one hour at the room temperature. The result is the article with fibrinogen solution. After this step is complete, the thickness of the article is 0.5 mm to 1 mm. The thickness of the fibrinogen layer is 5 μm to 10 μm. The relative weight of the fibrinogen to that of the shells was 0.1% w/w.

Experiment 8 to Experiment 10: Adding artificial polymer having polyvinyl alcohol (PVA), polyaniline (PANI), polyethylenimine (PEI) to the article.

Eighth Experiment: A solution of polyaniline (PANI) having a concentration of 2,000 μm to 6,000 ppm was prepared. The medical dressing article was submerged in this PANI solution in 10 minutes at the room temperature. The product obtained was the article with PANI solution. The article has a thickness of 0.5 mm to 1 mm. The thickness of the PANI solution is 5 μm to 10 μm. The relative weight of the PANI versus that of the crab shell was 0.1% w/w.

Ninth Experiment: A solution of polyvinyl alcohol (PVA) having a concentration of 10% to 30% was prepared. The PVA was sprayed onto the 2 cm by 2 cm wound care gauze at a spraying speed 0.5 ml/min for one hour at the temperatures from 50° C. to 80° C. The product obtained was the article with PVA solution. The article has a thickness of 0.5 mm to 1 mm. The thickness of the PAV solution is 0.5 mm. The relative weight of the PAV versus that of the crab shell was 5% w/w.

Tenth Experiment: A solution of polyethyleneimine (PEI) having a concentration of 2,000 μm to 6,000 ppm was prepared. The medical dressing article was slowly submerged in this PEI solution in 1 mm/min at the room temperature. The product obtained was the article with PEI solution. The article has a thickness of 0.5 mm to 1 mm. The thickness of the PEI solution is 10 μm. The relative weight of the PEI versus that of the crab shell was 0.1% w/w.

Experiment 11 to Experiment 13: Adding medicinally active inorganic ingredients including solution of nano silver nano particles, cupric oxide nano particles (CuO NP), and zinc nano particles (ZnNP) to the article.

Experiment 11: A solution of silver nano particles (AgNP) having a concentration of 1 ppm to 1000 ppm was prepared. The article was dipped into this nano Zinc solution in 30 minutes at the room temperature. The product obtained was the article covered with silver nano particles. The amount of silver nano particles (AgNP) cannot exceed 30 ppm. The article with AgNP has a thickness of 0.5 mm to 1 mm.

Experiment 12: A solution of cupric oxide nano particles (CuO NP) having a concentration of 1 ppm to 30 ppm was prepared. The article was covered with this cupric oxide nano particle (CuO NP) by spraying at a rate of 0.5 ml/minute for 30 minutes at the room temperature. The product obtained was the article covered with cupric oxide nano particle. The amount of cupric oxide nano particles (CuO NP) cannot exceed 30 ppm in the final product. The article imbued with CuO NP has a thickness of 0.5 mm to 1 mm.

Experiment: A solution of zinc nano particles (ZnNP) having a concentration of 1 ppm to 30 ppm was prepared. The article was dipped into this zinc nano particle solution at a rate 1 mm/minute in 30 minutes at the room temperature. The product obtained was the article covered with zinc nano particles (ZnNP). The amount of zinc nano particles (ZnNP) cannot exceed 30 ppm of the final product. The article with AnNP has a thickness of 0.5 mm to 1 mm.

Experimental Results

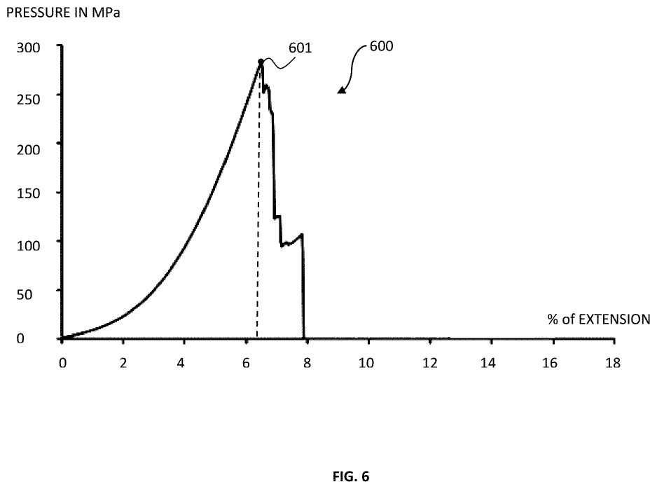

Experiment 14: Next referring to FIG. 6, a graph 600 presents the mechanical strength of the medical dressing article after being treated with the base solution and the acid solution as described above. The article has a dimension of 10 mm by 40 mm is tested by a TA XYplus, Stable Micro System, made in USA. The article is clamped tightly at the two ends and stretched out by a single axis force. The vertical axis represents the applying pressure to the article in Mpa. The horizontal axis represents the % stretch of the article. The maximum length of the article stretched out to 10 mm±4.2%. Demineralization and deproteinization made the membrane softer and thinner, but its tensile strength of 305.7±29. 9 MPa as shown at a peak 601 was more than 30 times higher than previously developed chitin membranes. After peak 601 or tensile strength, the article starts to lose elasticity. That is, the article is still stretched out even the pressure decreases.

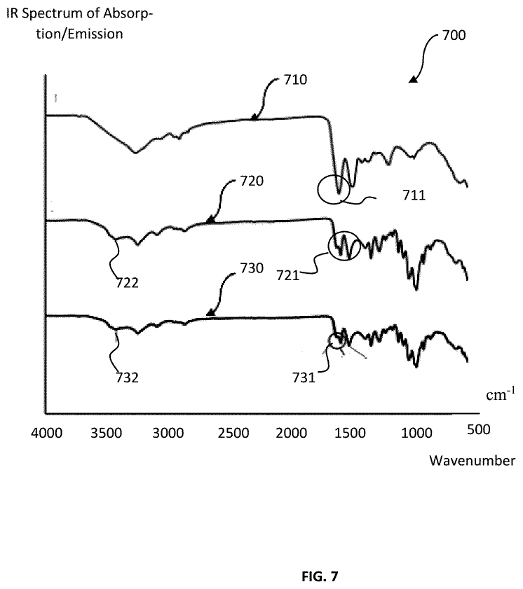

Experiment 15: Next referring to FIG. 7, a Fourier Transform Infrared spectroscopy (FT-IR) graph 700 of untreated, deproteinized, demineralized medical dressing articles is illustrated. A FT-IR scope by Spectrum GX, PerkinElmer, USA was used to measure the infrared spectrum of crab shells in three different conditions: a graph 710 represents the infrared spectrum of an untreated crab shell, a graph 720 represents the FTIR infrared spectrum of a deproteinized crab shell, and a graph 730 measures the FTIR infrared spectrum of a demineralized crab shell. Graph 710 showed characteristic peaks of β-chitin which is an undivided amide band 711 at 1640 cm⁻¹ due to the hydrogen bonds between the chitin chains and an amide II stretch 712 at 1550 cm⁻¹. However, after treatment, both graphs 720 and 730 show that respective amide I bands were split into two smaller peaks 721 and 731 at 1,622 cm⁻¹ and 1,645 cm⁻¹, indicating the conversion to α-chitin. Additionally, the intensity of an —OH and —NH stretch 722 and 732 in the region 3,000-3,500 cm⁻¹ decreased in post-treatment, further confirming the change in the intramolecular hydrogen bonds and chitin's conformation [7].

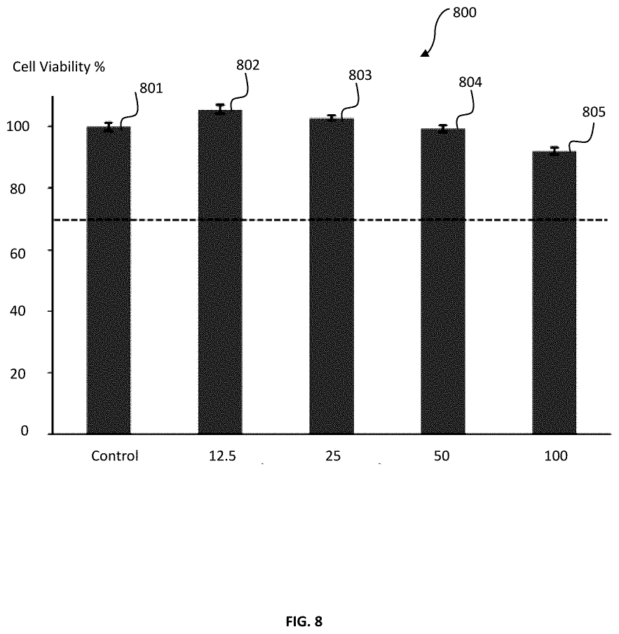

Example 15: Referring to FIG. 8, the cytotoxicity test was performed according to ISO 10993-1:2018 guidelines. The extract was obtained by dipping crab shells in cell culture medium for 24 hours at a concentration of 6 cm2/mL. This solution is 100% crab shell extract. The crab shells are then removed, and the remaining solution is made to different concentrations of 50%, 25%, and 12.5% respectively. Fibroblasts of L929 mice were cultured in a 96-well plate at a density of 104 cells/well, medium volume of 100 L/wells for 24 hours. Then, the culture medium was removed and 100 L of solution was added. The extraction membrane diluted with concentrations of 100% represented by a bar graph 801, 50% represented by a bar graph 802, 25% represented by a bar graph 803, 12.5% represented by a bar graph 804, 0% represented by a bar graph 805 respectively. The cells were incubated overnight at 37° C. Then 100 μL of cell culture medium with 11 resazurin (0.02 gh/mL) was added to each well and incubated for additional 4 hours. The culture plate was read at Ex/Em wavelength 530/590 nm using a multi-mode reader (Varioskan, Thermo Fisher, USA). Fluorescence signal of solution with cells cultured in medium Normal field was considered to have 100% survival (control). Each test was performed in four times. According to FIG. 8, at all concentrations of the extract crab shells, cell viability of bar graphs 802-805 is over 90% higher than the threshold of viability 70%. Therefore, crab shells prepared in accordance with method 100 of the present invention are non-toxic to living cells.

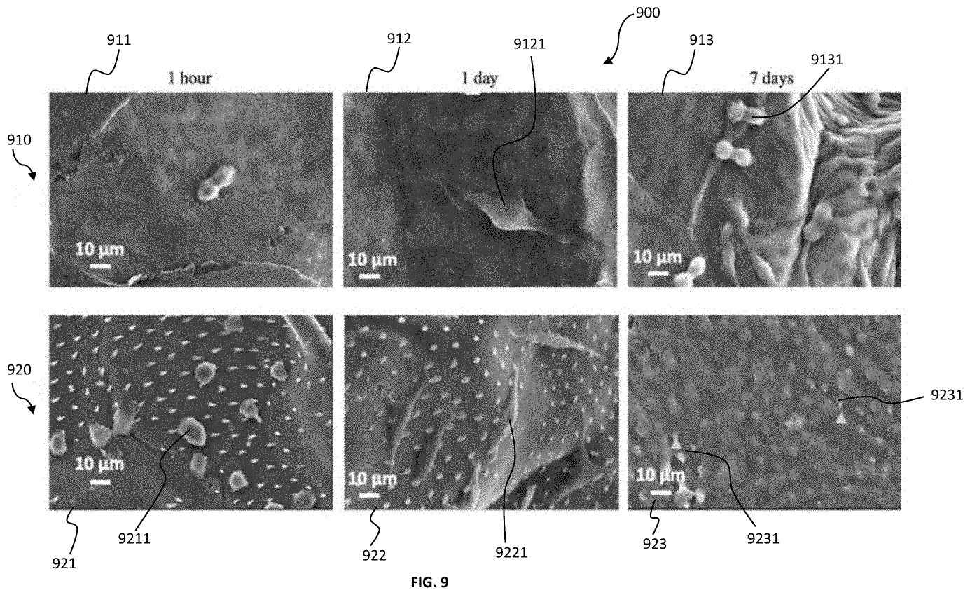

Experiment 17: Referring to FIG. 9, micrographs of cell viability test on a wound with and without the treatment of the medical dressing articles of the present invention after one hour, one day, and seven days are illustrated. Micrographs 910 represent the wound conditions without the treatment of medical dressing article 400C after 1 hour 911, 1 day 912, and one week 913. Micrographs 920 represent the wound conditions being treated by medical dressing article 400C after 1 hour 921, 1 day 922, and one week 923. According to FIG. 9, after one hour, micrograph 911 shows nothing happened in the wound without treatment. On the contrary, micrograph 921 shows cells 9211 started to appear. After 1 day, without the treatment of medical dressing article 400C, micrograph 912 shows that wound started to heal with the appearance of cell 9121. In micrograph 922, with the treatment of medical dressing article 400A, cells 9221 grew and spread all over the inner surface of the wound. As observed, cells 9221 were more attached to the lateral surface; especially, in the spines on the membrane containing arrays of microscopic needles 221b. Differences in cell proliferation and cell viability were more conspicuous after seven days. Micrograph 913—wound without treatment of medical dressing article 400C—shows that cells 9131 adhered to the outside become more obvious only after 7 days. However, the surface of wound without treatment was not even, showing the tendency of scar formation. After 7 days, micrograph 923 shows that cells 9231 proliferated more on the inner surface of the membrane of the wound, covering the entire surface and spikes. The surface of the wound became smooth and healed better without leaving a scar. Experiment 17 showed that wound with the treatment of medical dressing article 400C of the present invention and with arrays of microscopic needles 221b promoted cells growth and healed better than wound without treatment. After seven days, the surface of the wound with the treatment with the treatment of medical dressing article 400C was smooth and proliferated with cells 9231. While the surface of wound without treatment was rough and slow in healing with the presence of only a few cells 9131.

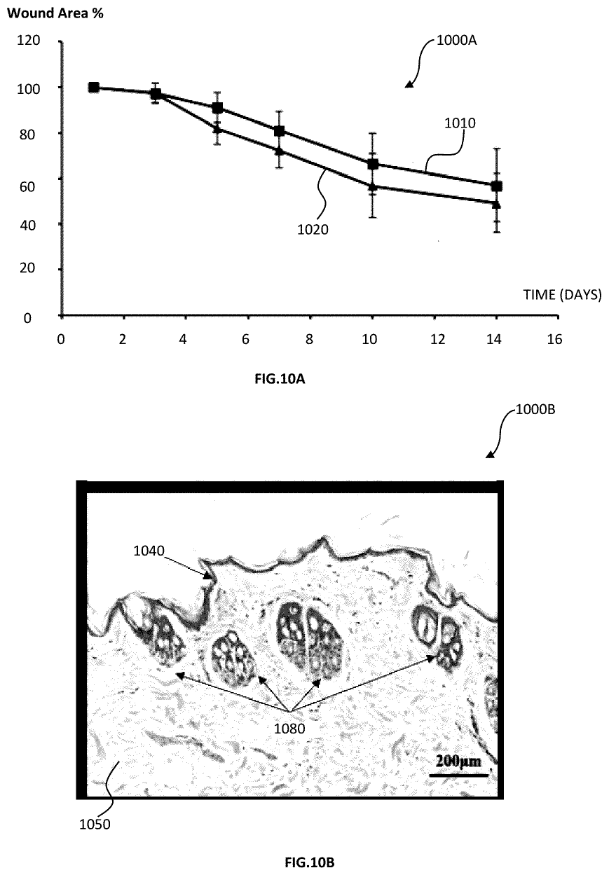

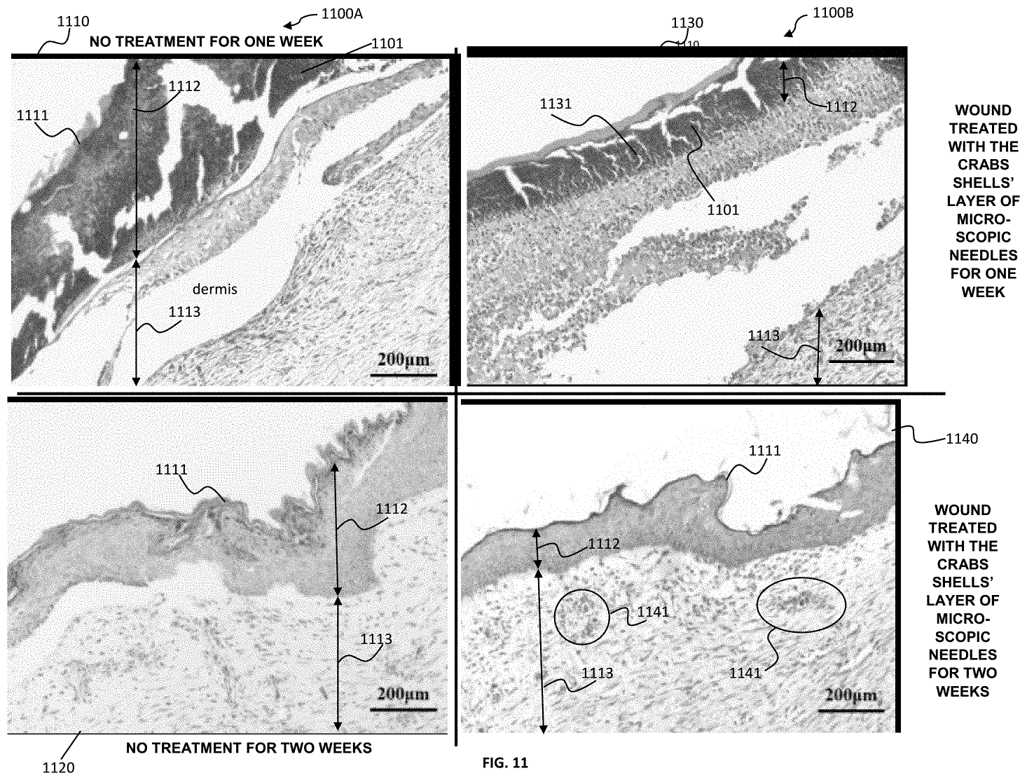

Experiment 18: Referring to FIG. 10A-FIG. 10B, and FIG. 11 which demonstrate experiments on would healing ability of wound dressing articles 400A to 400C of the present invention in comparison to the control wound treatment without any gazes for 14 days. The comparison of wound healing ability of medical dressing 400A/400B/400C made from the soft crab shells with arrays of microscopic needles 221b as shown in FIG. 2B is evaluated on a rabbit. The initial wound was created with an area of 3 cm×3 cm square. A treated group was designated as group 1020 and the untreated (control) group was designated as group 1010. The percentage (%) of wound reduction in size represented by the Y-axis of a graph 1000A was calculated from the originally generated wound area. FIG. 10A shows that on the 5^{th} day after the rabbit was wounded, a thin scab appeared on the wound of untreated group 1010, and cotton gauze was completely adhered to the wound. On the 14^{th} day, scales of both groups 1010 and 1020 fell. There were no signs of inflammation of both groups. After 14^{th} day, the size the wound areas of both groups 1010 and 1020 decreased by 50% as compared to the original wound. Results showed that when observed with the naked eyes, the wound with gauze in group 1020 healed faster than those without and do not have any infections as compared to those without treatment 1010.

Referring now to FIG. 10B, a histological staining diagram 1000B of a normal skin is shown. An epidermis layer 1040, a dermis layer 1050, and blood vessels 1080 of the normal skin are shown in diagram 1000B. There were no gaps or punctures in epidermis layer 1040. Blood vessels 1050 are intact and healthy. The color of dermis layer 1050 showed no staining (bruises).

Finally, referring to FIG. 11, micrographs 1100A and 1100B of histological staining tests of treatment a first group untreated and a second group treated with medical dressing article 400A respectively are shown. The histological staining test performed on the control group after 7 days are shown in a micrograph 1110 and after 14 days in a micrograph 1120. This test was also performed on second group treated with medical dressing article 400A after 7 days in a micrograph 1130 and 14 days in a micrograph 1140. After 7 days, as shown in micrograph 1100, epidermis layers 1112 of untreated wound 1110 was much damaged more irregularly as compared to that of the treated wound in micrograph 1130. This may be due to the spikes 221b on the inner surfaces of the molting crab shells that provide adhesion points for epidermal cells to proliferate. Moreover, the density of neutrophils and macrophases 1101 infiltrating the epidermis layer 1112 was regulated and treated less, indicating that membrane-treated wounds triggered less immune responses than the treated wounds. This proved that medical dressing articles of the present invention had good biocompatibility properties.

After 14 days, as shown in micrograph 1130, the differences between the two groups 1120 and 1140 were increasingly evidenced. As shown in micrograph 1120, the control wound still had fecal matters between epidermis layer 1112 and dermis layer 1113. On the contrary, as shown in micrograph 1140, these two layers were completely reattached in the treated samples. In addition, dermis layer 1113 formed blood vessels 1141. While the dermis layer 1113 did not develop these structures in the control group as shown in micrograph 1120.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

### DESCRIPTION OF NUMERALS

200A SEM images of the exterior surface of the crab shell
200B SEM images of the interior surface of the crab shell
201 mesopores in the exterior side of the untreated crab shell
202 micro ridges in the exterior side of the untreated crab shells
203 exterior surface mesopores of the deproteinized crab shell
204 exterior side micro ridges of the deproteinized crab shell
206 exterior surface mesopores of the dematerialized crab shell
207 exterior surface micro ridges of the dematerialized crab shell
221b microscopic needles in the interior side of crab shells
222b chitinous layer
401a polycaprotoner (PCL) impermeable layer
401b PCK impermeable layer
402a Microscopic needles layer obtained from method 100
402b PCL edge protectors
402c Needles layer deposited upon a PCL and poloxamer
403b optional bioadhesives
404b needles layers on a PCL layer with edge protectors
600 graph of tensile test
601 tensile strength of the layer of microscopic needles
700 FT-IR
710 FT-IR of untreated crab shell
711 amide band at 1640 cm⁻¹
720 FT-IR of deproteinized crab shell
721 peak at 1,622 cm⁻¹ indicating α-chitin conversion
722 intensity of an —OH and —NH stretch
730 FT-IR of demineralized crab shell
731 peak at 1,645 cm⁻¹ indicating α-chitin conversion
732 intensity of an —OH and —NH stretch
800 Cell viability test
801 control group
802 12.5% group
803 25% group
804 50% group
805 100% group
900 wound treatment tests with and without article
910 wound treatment without article
911 wound treatment without article after 1 hour
912 wound treatment without article after 1 day
9121 cell growth after 1 day of wound without article
913 wound treatment without article after 7 days
9131 cells growth after 7 days of wound without article
920 wound treatment with the article of the present invention
921 wound treatment with the article after 1 hour
9212 cell growth after 1 hour with the article
922 wound treatment with the article after 1 day
9221 cell growth after 1 day with the article
931 wound treatment with the article after 7 days
9311 cell growth after 7 days with the article
1000A wound area reduction %
1010 wound area reduction % without article
1020 wound area reduction % with article
1040 epidermis layer
1050 dermis layer
1080 blood vessels
1101 neutrophils and macrophases
1110 wound without treatment for one week
1111 strata corneum
1112 epidermis layer
1113 dermis layer
1120 wound without treatment for two weeks
1130 wound treated by the membrane for one week
1131 chitin layer
1140 wound treated by the membrane for two weeks
1141 blood vessels

### REFERENCES

[1] S. Islam, M. A. R. Bhuiyan, M. N. Islam, Chitin and chitosan: structure, properties and applications in biomedical engineering, J. Polym. Environ. 25 (3) (2017) 854-866, https://doi.org/10.1007/s10924-016-0865-5.
[2] R. Singh, M. P. Chacharkar, A. K. Mathur, Chitin membrane for wound dressing application—Preparation, characterisation and toxicological evaluation, Int. Wound J. (2008). https://doi.org/10.1111/j.1742-481X.2008.00482.x.
[3] E. Khor, L. Y. Lim, Implantable applications of chitin and chitosan, Biomaterials 24 (13) (2003) 2339-2349, https://doi.org/10.1016/S0142-9612(03)00026-7.
[4] B. Hastuti, Mudasir, D. Siswanta, Triyono, The synthesis of carboxymethyl chitosan-pectin film as adsorbent for lead (II) metal, Int. J. Chem. Eng. Appl. (2013) 349-353, https://doi.org/10.7763/IJCEA.2013.V4.323. [5] D. Revi, V. P. Vineetha, J. Muhamed, A. Rajan, T. V. Anilkumar, Porcine cholecyst-derived scaffold promotes full-thickness wound healing in rabbit, J. Tissue Eng. (2013). https://doi.org/10.1177/2041731413518060.
[5] D. Revi, V. P. Vineetha, J. Muhamed, A. Rajan, T. V. Anilkumar, Porcine cholecyst-derived scaffold promotes full-thickness wound healing in rabbit, J. Tissue Eng. (2013). https://doi.org/10.1177/2041731413518060.
[6] R&D Systems, Protocol for the Preparation and Fluorescent IHC Staining of Frozen Tissue Sections: R&D Systems, (n.d.). https://www.rndsystems.com/resources/protocols/protocol-preparation-and-fluorescent-ihc-staining-frozen tissue-sections (accessed Jan. 21, 2021).
[7] D. Ciolacu, J. Kovac, V. Kokol, The effect of the cellulose-binding domain from Clostridium cellulovorans on the supramolecular structure of cellulose fibers, Carbohydr. Res. 345 (5) (2010) 621-630, https://doi.org/10.1016/j.carres.2009.12.023.
[8] S. ichi Aiba, M. Izume, N. Minoura, Y. Fujiwara, Preparation and properties of chitin membranes, Carbohydr. Polym. 5 (1985) 285-295. https://doi.org/10.1016/0144-8617(85)90036-0

What is claimed is:

1. A medical dressing article, comprising:
a polymer substrate made of polycaprolactone and/or poloxamer; and
a layer containing arrays of microscopic needles, deposited on top of said polymeric substrate, elicited from (a) deproteinizing crab shells with a 0.5 mole to 1.0 mole base solution at 25° C. to 50° C. for 4 hours to six hours, and then (b) demineralizing said deproteinized crab shells with a 0.5 mole to 1.0 mole acid solution 25° C. to 50° C. for 4 hours to six hours, and then finally to (c) adding active medical ingredients.

2. The article of claim 1 wherein said crab shells (exoskeletons) which are new soft shells collected from molting crabs.

3. The article of claim 1 wherein a thickness of said layer containing arrays of microscopic needles is from 0.5 mm to 1 mm.

4. The article of claim 1 wherein said medical active ingredients are selected from Hyaluronic Acid (C₁₄H₂₁NO₁₁)ₙ, Chondroitin sulfate, and heparin (C₁₂H₁₉NO₂₀S₃).

5. The article of claim 1 wherein said medicinally active ingredients are selected from polysaccharide, artificial polymer, and a protein mixture.

6. The article of claim 5 wherein said polysaccharide is selected from hyaluronic acid, chondroitin sulfate, dextrane, alginate, and heparin.

7. The article of claim 8 wherein said protein mixture is selected from sericin, fibrin, fibrinogen, collagen, gelatin, and gelatin methacryloyl.

8. The article of claim 5 wherein said artificial polymer further comprises, poly(L-lactic acid) (PLLA-poly(L-lactic acid)), poly (L-lactic acid-coglycolic acid) (PLGA-poly(L-lactic acid-co-glycolic acid)), poyetyle glycol (PEG-polyethyl glycol), polyvinyl alcohol (PVA-polyvinyl alcohol), polyaniline (PANI-polyaniline), and polyethylenimine (PEI-polyethylenimine).

9. The article from claim 1 wherein said medically active ingredients are selected from a ZnO, CuO, copper alloy, nano silver, nano copper, and nano zinc.

10. The article of claim 4 wherein said base solution is selected from sodium hydroxide (NaOH) and potassium hydroxide (KOH).

11. The article of claim 1 wherein said acid solution is hydrochloride acid (HCl)

12. The article of claim 1 wherein said polycaprolactone comprised fibers having a dimer of 0.7 μm to 2.9 μm mixed with poloxamer having a content of 30%.

13. The article of claim 8 wherein said treatment layer has a thickness of 5 μm to 10 μm.

14. The article of claim 8 wherein said protein mixture has a percentage weight of 0.1 percent to 5 percent of a total weight of said crab shells.

15. A method of manufacturing a medical dressing article, comprising:
(a) preparing raw crustacean shells (exoskeletons) having a first predetermined percentage weight (% w/w);
(b) deproteinizing by treating said crustacean shells with a 0.5 mole to 1 mole base solution at a temperature of 25° C. to 50° C. for 4 hours to 6 hours;
(c) demineralizing said deproteinized crustacean shells obtained from step (b) by treating with an 0.5 mole to 1 mole acid solution at a temperature of 25° C. to 50° C. for 6 hours to 12 hours in order to obtain a layer containing arrays of microscopic needles; and
(d) depositing said layer containing arrays of microscopic needles directly on a polymeric substrate so that a side having microscopic needles make direct contact with a wound.

16. The method of claim 15 further comprising a step of: (e) adding medical active ingredients having a second predetermined percentage weight (% w/w) to said layer containing arrays of microscopic needles, wherein said medical active ingredients are comprised of polysaccharide, artificial polymer, and protein.

17. The method of claim 16 wherein said medical active ingredients are selected from hyaluronic acid, chondroitin sulfate, dextrane, alginate, and heparin, sericin, fibrin, fibrinogen, collagen, gelatin, gelatin methacryloyl, poly(L-lactic acid) (PLLA-poly(L-lactic acid)), poly (L-lactic acid-coglycolic acid) (PLGA-poly(L-lactic acid-co-glycolic acid)), poyetyle glycol (PEG-polyethyl glycol), polyvinyl alcohol (PVA-polyvinyl alcohol), polyaniline (PANI-polyaniline), and polyethylenimine (PEI-polyethylenimine).

18. The method of claim 15 wherein said polymeric substrate further comprises a polycaprolactone and poloxamer having a 30 percentage weight (% w/w).

19. The method of claim 18 wherein said layer containing arrays of microscopic needles is deposited directly on and surrounded by said polycaprolactone and poloxamer so as to prevent water from entering said wound from the outside environment and so as said wound makes direct contact with said arrays of microscopic needles.

20. The method of claim 15 wherein said base solution is selected from sodium hydroxide (NaOH) and potassium hydroxide (KOH) and said acid solution is hydrochloride acid (HCl).
